(19) United States
(12) Patent Application Publication
(10) Pub. No.: US 2025/0120849 A1
(43) Pub. Date: Apr. 17, 2025

(54) INSERTION SYSTEM FOR DELIVERY OF ACTIVE PHARMACEUTICAL AGENTS INSIDE THE EYE

(71) Applicant: GLAUKOS CORPORATION, Aliso Viejo, CA (US)

(72) Inventors: Alex Milewski, Aliso Viejo, CA (US); Richard Martin, Aliso Viejo, CA (US); Charles Kalina, Aliso Viejo, CA (US); Robert Balsamo, Aliso Viejo, CA (US); Douglas Crimaldi, Aliso Viejo, CA (US); John Muri, Aliso Viejo, CA (US); Tyler Brown, Aliso Viejo, CA (US)

(21) Appl. No.: 18/913,142

(22) Filed: Oct. 11, 2024

Related U.S. Application Data
(60) Provisional application No. 63/590,204, filed on Oct. 13, 2023.
(60) Provisional application No. 63/642,441, filed on May 3, 2024.

(51) Int. Cl. A61F 9/00

(52) U.S. Cl. CPC A61F 9/0017

(57) ABSTRACT

A delivery system includes an insertion device and an implant. The insertion device includes a body comprising a push button, a pusher wire, a backbone and a pushing mechanism. The pusher wire comprises a first end and a second end. The second end of the pusher wire is coupled to the pushing mechanism. The insertion device further includes an inserter tip comprising a needle. The inserter tip is configured to affix to the body. The implant is configured to be loaded into the insertion device. Upon depression of the push button, the pushing mechanism is activated and the pushing mechanism pushes the pusher wire into the implant and through the needle at a constant velocity and a predefined distance.

### PRIORITY CLAIM AND CROSS-REFERENCE TO RELATED APPLICATIONS

This application claims priority to and benefit of U.S. Provisional Application No. 63/590,204, filed Oct. 13, 2023, and U.S. Provisional Application No. 63/642,441, filed May 3, 2024, the entire contents of which are hereby incorporated by reference in their entirety including all tables, figures, and claims.

### FIELD

The present disclosure relates generally to the delivery of pharmaceuticals to the eye via insertion systems.

### BACKGROUND

One method of pharmaceutical delivery to eye, and in particular to the retina, is to inject the pharmaceutical in a pill or tablet form (hereafter referred to more generally as pharmaceutical implant or implant) into the posterior chamber of the eye. Consistent delivery conditions are critical to ensure proper placement of the pharmaceutical implant while avoiding undesirable contact with certain anatomical features within the eye. One way of ensuring consistent delivery conditions is by the use of a hand piece that provides the doctor or surgeon with an easily controllable surgical procedure.

Current commercially available devices may require manual activation by pushing a button for implant delivery. Pushing the button generates an implantation velocity onto the implant, but this implantation velocity is largely dependent on the speed and force applied to the button. For example, if a surgeon depresses the button too quickly, the device may deliver the implant at an undesirably high implantation velocity, which can cause excessive delivery distance and may result in anatomical injury. Alternatively, if a surgeon depresses the button too slowly, the device may not deliver the implant with enough force to propel the implant fully into the eye; this can result in unintentional implant removal (e.g., via suction created by the manual device as it is withdrawn from the implantation location).

As one can appreciate, the velocity variation inherent in these widely available push button devices is problematic for both the surgeon and the patient, as it may lead to undesirable implant placement and/or contact with delicate anatomical features within the eye. For these reasons, among others, new surgical hand pieces are needed that deliver an implant at a consistent velocity, to a consistent distance, independent of push button depression speed or force.

### SUMMARY

The present disclosure includes a hand piece with a push button activation and related features that ensure delivery of an implant at a consistent velocity to a consistent distance, independent of the speed or force applied to the push button. This consistent delivery paradigm allows the surgeon to focus on device positioning during the implantation event, as the surgeon knows that the device will deliver the implant at a consistent velocity to a consistent distance, thus avoiding both over-implantation and under-implantation. The systems disclosed herein generate a consistent delivery profile, including velocity, force, and displacement. The systems may implement mechanical stored energy through springs, flexures, gears, and the like.

In light of the disclosure herein, and without limiting the scope of the invention in any way, in a first aspect of the present disclosure, which may be combined with any other aspect listed herein unless specified otherwise, an insertion device for injecting an implant into an eye is provided. The insertion device may include a body and an insertion tip. The body may include a push button including a button, a first spring, and an arm extending from the button, The first spring may bias the arm and the button in an upward position. The body may further include a pusher wire, and a pushing mechanism comprising a shuttle and a second spring. The pusher wire may include a first end and a second, wherein the second end of the pusher wire is coupled to the shuttle. The inserter tip may include a needle, wherein the inserter tip is removably coupled to the body. Upon depression of the push button, the pushing mechanism may be configured to displace the pusher wire, such that a portion of the pusher wire translates through the needle.

In a second aspect of the present disclosure, which may be combined with any other aspect listed herein unless specified otherwise, the pusher wire may translate through the needle at a consistent velocity, wherein the consistent velocity is independent of a force applied to the push button via depression of the push button.

In a third aspect of the present disclosure, which may be combined with any other aspect listed herein unless specified otherwise, the body may further include a backbone, wherein the push button and the pushing mechanism are integrated with the backbone.

In a fourth aspect of the present disclosure, which may be combined with any other aspect listed herein unless specified otherwise, the shuttle includes a first groove and a second groove, wherein the first groove and the second groove are configured to receive a portion of the arm of the push button.

In a fifth aspect of the present disclosure, which may be combined with any other aspect listed herein unless specified otherwise, the insertion device may include a first inactivated state where the portion of the arm is in the first groove, a first active state where the portion of the arm disengages from the first groove, and a second inactivated state where the portion of the arm is in the second groove.

In a sixth aspect of the present disclosure, which may be combined with any other aspect listed herein unless specified otherwise, the insertion device may include a retaining member and an implant disposed within the needle. The retaining member may exert a force on the implant, holding the implant in place within the needle.

In a seventh aspect of the present disclosure, which may be combined with any other aspect listed herein unless specified otherwise, depression of the push button may cause the portion of the pusher wire to translate through the needle and into the implant, wherein a force exerted on the implant from the pusher wire is greater than the force from the retaining member, causing the implant to eject from the needle.

In an eighth aspect of the present disclosure, which may be combined with any other aspect listed herein unless specified otherwise, the implant includes a pharmaceutical for delivery to the eye.

In a ninth aspect of the present disclosure, which may be combined with any other aspect listed herein unless specified otherwise, an insertion tip configured to be removably coupled to an insertion device for injecting an implant into an eye is provided. The insertion tip may include a needle and a cover for receiving the needle. The cover may include one or more slots, and the one or more slots may include a notch on a side surface of the slot. The one or more slots may be configured to secure the insertion tip to the insertion device.

In a tenth aspect of the present disclosure, which may be combined with any other aspect listed herein unless specified otherwise, the needle includes a double beveled edge.

In an eleventh aspect of the present disclosure, which may be combined with any other aspect listed herein unless specified otherwise, the inserter tip may include the implant configured to be injected into the eye.

In a twelfth aspect of the present disclosure, which may be combined with any other aspect listed herein unless specified otherwise, the implant is disposed within the needle.

In a thirteenth aspect of the present disclosure, which may be combined with any other aspect listed herein unless specified otherwise, the inserter tip may further include a retaining member configured to exert a force on the implant into an internal surface of the needle.

In a fourteenth aspect of the present disclosure, which may be combined with any other aspect listed herein unless specified otherwise, the retaining member includes a distal end portion, a proximal end portion, and an elongate member between the distal end portion and the proximal end portion, wherein the distal end portion is configured to exert the force on the implant into the internal surface of the needle.

In a fifteenth aspect of the present disclosure, which may be combined with any other aspect listed herein unless specified otherwise, the proximal end portion of the retaining member is configured to anchor the retaining member to the needle, and the elongate member is disposed within the needle.

In a sixteenth aspect of the present disclosure, which may be combined with any other aspect listed herein unless specified otherwise, the implant is a first implant, and the insertion tip further includes a second implant.

In a seventeenth aspect of the present disclosure, which may be combined with any other aspect listed herein unless specified otherwise, the first implant and the second implant are disposed in series within the needle.

In an eighteenth aspect of the present disclosure, which may be combined with any other aspect listed herein unless specified otherwise, a method of inserting an implant into an eye is provided. The method may include inserting a needle of an insertion device into the eye, wherein the insertion device comprises a body removably coupled to an insertion tip, wherein the body comprises a push button, a pusher wire, and a pushing mechanism and wherein the insertion tip comprises a needle. The method may further include depressing the push button of the insertion device, such that the implant is delivered through the needle of the insertion tip and into the eye at a consistent velocity.

In a nineteenth aspect of the present disclosure, which may be combined with any other aspect listed herein unless specified otherwise, the method may include loading the implant into a distal end of the insertion device prior to inserting the needle into the eye.

In a twentieth aspect of the present disclosure, which may be combined with any other aspect listed herein unless specified otherwise, the method may include affixing the insertion tip to the body prior to inserting the needle of the insertion device.

In a twenty-first aspect of the present disclosure, which may be combined with any other aspect listed herein unless specified otherwise, a delivery system is provided. The delivery system may include an insertion device including a body and an inserter tip. The body may include a push button, a pusher wire, a pushing mechanism, and a backbone, wherein the pusher wire includes a first end and a second end and wherein the second end of the pusher wire is coupled to the pushing mechanism. The inserter tip may include a needle, wherein the inserter tip is removably coupled to the body. The delivery system may further include an implant configured to be loaded into a distal end of the insertion device. Upon depression of the push button, the pushing mechanism is activated, and the pushing mechanism displaces the pusher wire, such that a portion of the pusher wire and the implant translate through the needle.

In a twenty-second aspect of the present disclosure, which may be combined with any other aspect listed herein unless specified otherwise, the first end of the pusher wire translates through a tip of the needle at a predefined distance, defined by a distance between the tip of the needle and the first end of the pusher wire.

In a twenty-third aspect of the present disclosure, which may be combined with any other aspect listed herein unless specified otherwise, the predefined distance is between 0.75 mm and 1.25 mm.

In a twenty-fourth aspect of the present disclosure, which may be combined with any other aspect listed herein unless specified otherwise, the implant translates out of the inserter tip and into an eye at a consistent velocity.

In a twenty-fifth aspect of the present disclosure, which may be combined with any other aspect listed herein unless specified otherwise, the consistent velocity is between 1000 mm/s and 2000 mm/s.

In a twenty-sixth aspect of the present disclosure, which may be combined with any other aspect listed herein unless specified otherwise, the consistent velocity is independent of a force applied to the push button via depression of the push button.

In a twenty-seventh aspect of the present disclosure, which may be combined with any other aspect listed herein unless specified otherwise, the delivery system further includes a cap configured to cover the needle.

In a twenty-eighth aspect of the present disclosure, which may be combined with any other aspect listed herein unless specified otherwise, the pushing mechanism includes a spring and a shuttle.

In a twenty-ninth aspect of the present disclosure, which may be combined with any other aspect listed herein unless specified otherwise, the needle includes a double beveled edge.

In a thirtieth aspect of the present disclosure, which may be combined with any other aspect listed herein unless specified otherwise, the needle comprises a window.

In a thirty-first aspect of the present disclosure, which may be combined with any other aspect listed herein unless specified otherwise, the inserter tip includes a tube configured to surround a portion of the needle.

In a thirty-second aspect of the present disclosure, which may be combined with any other aspect listed herein unless specified otherwise, the push button comprises a button, a first arm extending from the button in a first direction, and a second arm extending from the button in a second direction, wherein the second arm is configured to hold the pushing mechanism in a compressed position.

Additional features and advantages of the disclosed devices, systems, and methods are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not necessarily have to have all of the advantages listed herein. Moreover, it should be noted that the language used in the specification has been selected for readability and instructional purposes, and not to limit the scope of the inventive subject matter.

### BRIEF DESCRIPTION OF THE DRAWINGS

Understanding that figures depict only typical embodiments of the invention and are not to be considered to be limiting the scope of the present disclosure, the present disclosure is described and explained with additional specificity and detail through the use of the accompanying figures. The figures are listed below.

FIG. 1 is a side view of an insertion system, according to an example embodiment of the present disclosure.

FIG. 2 is an isometric view of an inserter tip of the insertion system, according to an example embodiment of the present disclosure.

FIG. 3 is a cross-sectional view of an insertion device, according to an example embodiment of the present disclosure.

FIG. 4 is an isometric view of an insertion system, according to an example embodiment of the present disclosure.

FIG. 5 is an isometric view of an insertion tip before activation of a push button, according to an example embodiment of the present disclosure.

FIG. 6 is a cross-sectional view of an insertion tip before activation of the push button, according to an example embodiment of the present disclosure.

FIG. 7 is an isometric view of the insertion tip after activation of the push button, according to an example embodiment of the present disclosure.

FIG. 8 is a cross-sectional view of an insertion tip after activation of the push button, according to an example embodiment of the present disclosure.

FIG. 9 is an isometric view of an insertion device, according to an example embodiment of the present disclosure.

FIG. 10 is an isometric view of the insertion device with a protective cap, according to an example embodiment of the present disclosure.

FIG. 11 is an isometric view of the insertion device with a protective cap, according to an example embodiment of the present disclosure.

FIG. 12 is a side view of an inserter tip of the insertion device, according to an example embodiment of the present disclosure.

FIG. 13 is a top view depicting the interaction between the inserter tip and a body of the insertion device, according to an example embodiment of the present disclosure.

FIGS. 14A and 14B are isometric and cross-sectional views of an insertion tip with a retaining member, according to an example embodiment of the present disclosure.

FIG. 14C is an isometric view of a needle with a pivotable flap for retaining an implant according to an example embodiment of the present disclosure.

FIGS. 15A through 15C are isometric views illustrating a proximal end of the retaining member, according to example embodiments of the present disclosure.

FIG. 16A through 16C illustrates retaining members, according to example embodiments of the present disclosure.

FIG. 17 is a side view of the insertion device, with a portion of a housing removed for clarity, according to an example embodiment of the present disclosure.

FIG. 18 is a side view of the insertion device of FIG. 8, with a backbone removed for clarity, according to an example embodiment of the present disclosure.

FIGS. 19A through 19C are a side view of an insertion system, according to an example embodiment of the present disclosure.

FIGS. 20A and 20B are a bottom view and a close-up view of the internal components of an insertion device according to the present disclosure.

FIGS. 21A and 21B are cross-sectional views of insertion tips loaded with two implants.

FIGS. 22A through 22C are a side view of an insertion system for delivering multiple implants, according to an example embodiment of the present disclosure.

FIGS. 23A and 23B are a bottom view and a close-up view of the internal components of an insertion device for delivering multiple implants according to the present disclosure.

FIGS. 24A through 24F illustrate the process of the insertion device for deploying multiple implants according to an example of the present disclosure.

FIGS. 25A through 25D illustrate an insertion tip according to an example of the present disclosure.

FIG. 26 illustrates an insertion device packaged separately from the body.

FIG. 27 shows an insertion device constructed from a modular insertion tip and body.

### DETAILED DESCRIPTION

Additional features and advantages of the disclosed devices, systems, and methods are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein. Moreover, it should be noted that the language used in the specification has been selected for readability and instructional purposes, and not to limit the scope of the inventive subject matter.

The present disclosure relates generally to insertion devices and systems configured for delivery of an implant into the eye, such as the posterior chamber of the eye. Generally, the insertion device is configured to dissect the sclera of the eye and enter into the vitreous humor of the eye; the insertion device subsequently delivers the implant into the eye within the vitreous humor, into the retina, or into other desirable locations. In some embodiments, the insertion device is configured for intracameral injection, such as through the anterior chamber of the eye. The delivery location of the implant may be along Schlemm's canal, within the sclera, aqueous collector channels, episcleral veins, the uveoscleral outflow pathway, the supraciliary space, and/or the suprachoroidal space.

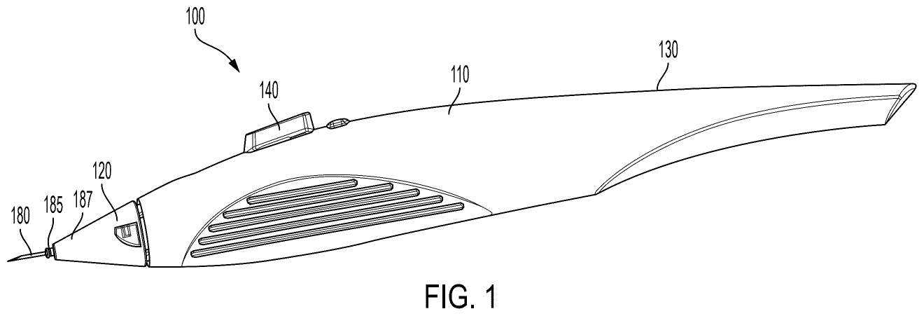

FIG. 1 shows a side view of an example insertion device 100, configured for delivery of an implant into the eye, such as the posterior chamber of the eye. In the illustrated embodiment, the insertion device 100 includes a body 110 and an inserter tip 120.

The inserter tip 120 can be separate from or, alternatively, affixed to the body 110. For example, the manufacturing process may involve packaging the body 110 and the inserter tip 120 separately for subsequent assembly by a user. Specifically, a user may affix the inserter tip 120 to the body 110 prior to performing an intended procedure, such as at a surgical site. The modular nature of the inserter tip 120 and the body 110 (as discussed in greater detail herein) allows different inserter tips to be used with the same body 110. Alternatively, the manufacturing process may involve affixing the inserter tip 120 to the body 110.

The inserter tip 120 may include a cover 187, a needle 180 extending from one end of the cover 187, and a tube 185 surrounding the outside of the needle 180. In one example, a user attaches the inserter tip 120 to the body 110, prior to loading the implant into the insertion device 100. For example, a surgeon may load one or more implants into the insertion device 100 through the end of the needle 180 extending from the cover 187. In an alternative example, the surgeon may load one or more implants into the inserter tip 120 before affixing the inserter tip 120 to the body 110. For example, the surgeon may load the implant into either side of the needle 180 prior to affixing the inserter tip 120 to the body 110. Preferably, the surgeon may load the implant into the distal end of the needle 180 to avoid damage of the needle and injury to the surgeon.

In another alternative example, the inserter tip 120 is preloaded with one or more implants during manufacturing. The manufacturing process may include inserting the implant into the distal end of the needle 180 and securing the implant within the needle 180. Preloaded inserter tips 120 may include a colored cover 187 depending on the type or dose of implant(s) or pharmaceutical(s) loaded into to the inserter tip 120. For example, one type of implant may have a blue cover indicating a first drug or dosage of drug, while another type of implant may have a red cover indicating a second different drug or dosage of drug. This helps prevent the surgeon from accidentally implanting the wrong pharmaceutical into the eye. In some examples, the inserter tip 120 is preloaded with one, two, or three implants (as described in greater detail herein). Further, it should be appreciated that different inserter tips 120, such as with different colors/dosages are compatible the same body 110. Namely, different implants and/or dosages of implants can be delivered using the same body 110. While inserter tips 120 may be a generally disposable component, body 110 is reusable within a given procedure (i.e., with multiple inserter tips).

Once the inserter tip 120 and the body 110 are secured to one another and the implant is loaded into the insertion device 100, a surgeon may grasp an external housing 130 of the body 110 and subsequently insert the needle 180 of device 100 into the eye. To activate the insertion device 100, a surgeon depresses the push button 140. By depressing push button 140, the implant is ejected from the needle 180 into the eye via mechanical components housed within body 110, as discussed in detail herein.

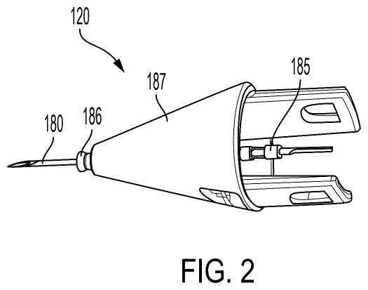

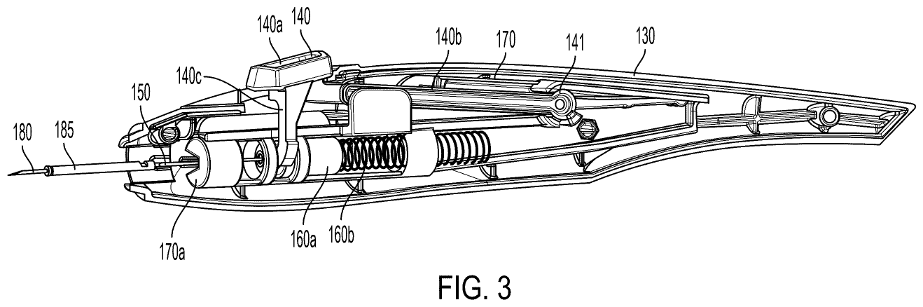

FIG. 2 shows an isometric view of the inserter tip 120 of the insertion device 100. As discussed, the inserter tip 120 may include a needle 180 and a cover 187. The cover 187 of the inserter tip 120 (not shown in FIG. 3) protects the components of the inserter tip 120. Additionally, as depicted, the cover 187 is configured to couple the inserter tip 120 to the body 110 of the insertion device 100. For example, the cover 187 of the inserter tip 120 may include one or more features configured to couple with one or more features of the body 110, such as the backbone of the body 110.

The needle 180 of the inserter tip includes a cannula bore, such as a channel extending along the length of the needle 180 configured for the implant to travel therethrough. The needle 180 may also be configured to incise intraocular tissue of the patient's eye. For example, the needle 180 may include a beveled edge, such as a single beveled edge or a double beveled edge. Preferably, the needle 180 includes a double beveled edge as shown in FIG. 2.

In one example, the needle 180 is a 22 gauge needle or smaller, for example, a 22 gauge needle to a 32 gauge needle. In a preferred embodiment, the needle is a 24 gauge needle. The needle 180 may be coated or uncoated. Specifically, the needle 180 may be coated, such as with MDX or other medical grade silicone coating fluids, to improve dispersion.

As depicted in FIG. 2, the inserter tip 120 may include tube 185 located on the exterior surface of the needle 180. In some embodiments, the tube 185 may be a band or collar. The tube 185 may increase the stiffness of the needle 180. The tube 185 may also secure the needle 180 inside the cover 187 of the inserter tip 120. The tube 185 allows the inserter tip 120 to be compatible with different needle sizes, pusher wire sizes, and/or implant sizes. For example, as long as the outer diameter of the tube 185 surrounding the needle 180 is consistent, different needle sizes, implants, and pusher wire sizes are compatible with the inserter tip 120. The tube 185 may also include an o-ring 186, silicon or any other elastomeric component. For example, the o-ring 186 may be disposed at one end of the tube 185 as illustrated.

Prior to use of the insertion device 100, one or more implants are loaded in the needle 180 of the inserter tip 120. To prevent movement of the implant after it is positioned within the needle 180, the o-ring 186 may be disposed on the outside of the needle 180 and through a window of the needle 180 to hold the implant into place within the needle 180. For example, the implant may generally have a diameter smaller than the internal diameter of the needle 180. This allows a user to easily load the implant into the needle 180; but, this configuration provides clearance between the implant and an internal surface of the needle 180. The o-ring 185 minimizes the clearance between the implant 200 and the internal surface of the needle 180.

The cover 187 may be used to protect the components of the inserter tip 120. Additionally, the cover 187 may be configured to couple to the body 110 of the inserter 100. For example, the cover 187 of the inserter tip 120 may include one or more features configured to couple with one or more features of the body 110, such as the housing 130 of the body 110. In the illustrated embodiment, the cover includes two slots configured to couple with two protrusions on the housing 120, securing the inserter tip to the inserter body 110. It can be appreciated that other securing mechanisms may be used to attach the inserter tip 120 to the body 110.

FIG. 3 shows a cross-sectional view of the insertion device 100 with the insertion tip 120 secured to the body 110 without the cover 187, for better visibility. The body 110 includes an external housing 130 configured to protect the components contained therein. The housing 130 may include an opening in which the push button 140 extends through, such as along a top side of housing 130. The housing 130 may include an elongated member configured to be grasped by a user. For example, the cross section of the housing 130 may change along the length of the housing 130, such as to enhance ergonomic functionality of the insertion device 100. Alternatively, the cross section of the housing 130 may be uniform along the length of the housing 130. In various example embodiments, the housing may include one or more ridges to improve a user's grip while holding the device. In some examples, the housing 130 may include two parts or halves which fit together to jointly form the housing 130. The two parts may be symmetrical, or the two parts may be asymmetrical. When symmetrical, it should be appreciated that housing 130, and more generally insertion device 100, is configured for ambidextrous use.

In this illustrated embodiment, the body 110 also includes a backbone 170, secured inside the housing 130 by internal components therein. For example, the backbone 170 may include one or more apertures in which bolts protruding from the inside of the housing fit, securing the backbone 170 to the housing 130.

The backbone 170 acts as an internal housing, supporting the internal mechanical components of the insertion device 100. As shown, the internal mechanical components include a push button 140, a pusher wire 150, and a pushing mechanism 160. The push button 140 and the pushing mechanism 160 may mount to the backbone 170 of the body 110. For example, the backbone 170 may include one or more features configured to couple with the push button 140 and/or the pushing mechanism 160. The use of backbone 170 may ensure that most of the precision engineering is isolated to one critical component of the insertion device 100.

The insertion device 100 may include a push button 140 configured to activate the insertion device 100 and deliver the implant into the eye. In some embodiments, the push button 140 can include a button 140a, a first arm 140b extending from the button 140a in a first direction, and a second arm 140c extending from the button 140a in a second direction. Generally, the button 140a is configured to be depressed by a user, such as via a user's thumb or finger. Before depression of the button 140a, the insertion device is generally disposed in an inactivated state as shown in FIG. 3. When the user depresses the button 140a, the insertion device activates, propelling the pushing mechanism 160 and pusher wire 150 forward, as will be discussed in more detail herein. In some embodiments, push button 140 and device 100 are configured for single-use actuation. Namely, once “activated,” pushing mechanism 160 and pusher wire 150 are propelled forward and subsequently may remain locked in this forward position.

As illustrated, the first arm 140b of the push button 140 extends along the length of the body 110 of the insertion device 100. A first end of the first arm 140b may attach to the button 140a, while the second end of the first arm 140b may engage with the backbone 170. In this illustrated embodiment, the second end of the first arm 140b is biased upward exerting a force on a portion of the backbone 170. Since the backbone 170 is in a fixed state secured to the housing 130, the force between the first arm 140b and the backbone 170 biases the button 140a upward. In some examples, in order to depress the button 140a, a user may exert a force on the button 140a greater than the load acting upon the first arm 140b.

In the illustrated embodiment, the first arm 140b includes a pivot point 141 upon which the push button 140 pivots when the user depresses the button 140a. The backbone 170 may be further configured to couple to the pivot point 141 of the first arm 140b. As illustrated, the pivot point 141 may be circular or another rounded shape.

The second arm 140c may extend from the button 140a and may engage with the pushing mechanism 160. In the inactivated state, the second arm 140c holds the pushing mechanism in a compressed state.

In the illustrated embodiment, the pushing mechanism 160 includes a shuttle 160a and a spring 160b, with the spring 160b coupled to a first end of the shuttle 160a. In the inactivated state, the spring 160b is compressed, storing potential energy. For example, one end of the spring 160b may contact and compress against a surface of the backbone 170.

The second end of the shuttle 160a may include one or more features configured to engage with a feature on the second arm 140c allowing the second arm 140c to hold the pushing mechanism 160 in a compressed state. In some embodiments, the shuttle 160a may include a groove around its circumference, and the second arm 140c may include a protrusion that fits into the groove of the shuttle 160a. When a user depresses the push button 140, the second arm 140c moves with respect to the shuttle 160a, disengaging the protrusion from the groove in the shuttle 160a, allowing the shuttle 162 to move in an axial direction, thus activating the pushing mechanism 160. Activation of the pushing mechanism 160 results in a (partial or full) release of stored mechanical energy via the compressed spring 160b. It can be appreciated that other mechanisms for storing potential energy can, alternatively, be used in place of the spring 160b. For example, the pushing mechanism 160 may include one or more springs, flexing components, and/or gears to store potential energy and, when activated, release (at least a portion of) the potential energy as kinetic energy.

The pusher wire 150 may couple to the pushing mechanism 160, for example, at the shuttle 160a. In some examples, the pusher wire 150 may be disposed within the shuttle 160a, such as through a channel in the shuttle 160a. Upon release of the second arm 140c from the shuttle 160a, the spring 160b forces the shuttle 160a forward, thus forcing the pusher wire 150 forward at a consistent velocity. It should be appreciated that the force with which the surgeon depresses the button 140a is irrelevant, so long as the force is sufficient to release second arm 140c from shuttle 160a. Thus, consistent velocity (via compressed spring 160b) is independent from depression force at button 140a. In some embodiments, the average velocity of the pusher wire 150 is between 100 mm/s and 8000 mm/s. Preferably, the average velocity of the pusher wire 150 is between 1000 mm/s and 2000 mm/s.

As shown in the illustrated embodiment, the backbone 170 may include a surface 170a, such as wall, in the path of the shuttle 160a to limit the distance the shuttle 160a travels after activation of the spring 160b. This may also limit the distance the pusher wire 150 travels, so that the pusher wire 150 travels a predefined distance. For example, when the inserter tip 120 is secured to the body 110, the pusher wire 150 may be disposed within the cannula bore of the needle 180. Upon depression of the push button 140, the pusher wire 150 may project through the needle 180 and past the tip of the needle 180 a predefined distance. In some embodiments, the pusher wire 150 moves past the tip of the needle 180 a predefined distance of 0.1 mm to 3 mm. Preferably, the predefined extension distance is 0.75 mm to 1.25 mm.

The pusher wire 150 may have any suitable shape or size. For example, the pusher wire may have a circular cross-section. The size of the pusher wire 150 can be optimized depending on the size of the needle 180 and the implant as to prevent air bubbles in the delivery of the implant. In one example, the pusher wire 150 is made of stainless steel and/or titanium. The pusher wire 150 can be colored, such as a color different from the needle 180, to differentiate between the needle 180. For example, pusher 150 may be neon green or another similar fluorescent color. Color differentiation helps users, such as surgeons or doctors, determine if the insertion device 100 has been activated. In some embodiments, the insertion device provides the user another indication that the insertion device has been activated, such as tactile or auditory feedback.

After a user secures the inserter tip 120 to the body 110, the cannula bore of the needle 180 receives at least a portion of the pusher wire 150. If a surgeon is loading the implant into the needle 180, the surgeon may push the implant in the needle 180 so that it contacts the pusher wire 150. Alternatively, the surgeon may load the implant into the distal end of the needle 180 before securing the inserter tip 120 to the body 110. The pusher wire 150 may interact with the implant upon interaction of the inserter tip 120 with the body 110. When the surgeon depresses the push button 140 and the pusher wire 150 traverses forward, the pusher wire 150 moves into contact with the implant. Accordingly, the pusher wire 150 propels the implant 200 out of the distal end of needle 180 and into a portion of the eye, such as the posterior chamber. The controlled distance the pusher wire 150 travels, and the related mechanics of spring 160b allow the implant to propel into the eye at a consistent velocity and to a consistent distance, independent of the force with which the surgeon depresses the push button 140. The velocity of the implant is intended to be high enough to ensure adequate implantation depth; for example, this ensures that removal of device 100 from the eye does not create suction causing removal of the implant itself.

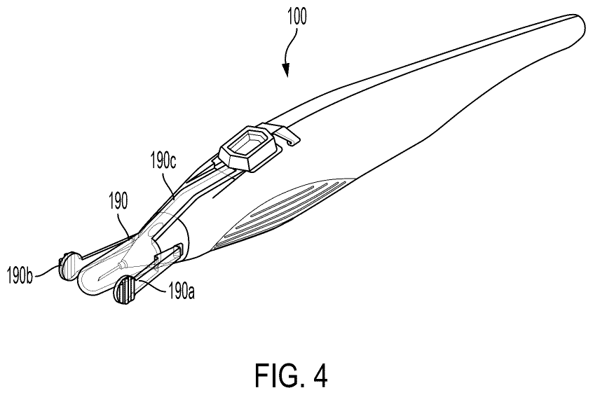

As shown in FIG. 4, in some examples, the insertion device 100 includes a protective cap 190 configured to protect the needle 180 and the inserter tip 120 during transportation and handling. Specifically, the protective cap 190 can provide protection to the user when affixing the inserter tip 120 to the body 110. The protective cap 190 can be transparent or comprise an optical window to ensure presence of the implant without removal of the cap 190. The user removes the protective cap 190 from the insertion device 100, prior to use. The protective cap 190 can be affixed to the inserter tip 120 or the body 110 by any fastening means.

The protective cap 190 of the inserter tip 120 may act as a locking mechanism to prevent push button 140 from inadvertent depression during transportation and handling. For example, as shown in FIG. 4, the protective cap 190 may include an arm 190c that rests under the push button 140, preventing the push button from being depressed. In some embodiments, the protective cap 190 can also include one or more tabs 190a that engage with grooves along housing 130 or inserter tip 120. The user may squeeze or separate the tabs 190a for easier removal of the protective cap 190 from the inserter tip 120. Additionally, the tabs 190a of the protective cap 190 can include grooves or protrusions for improving the grip of the user during removal of the cap 190.

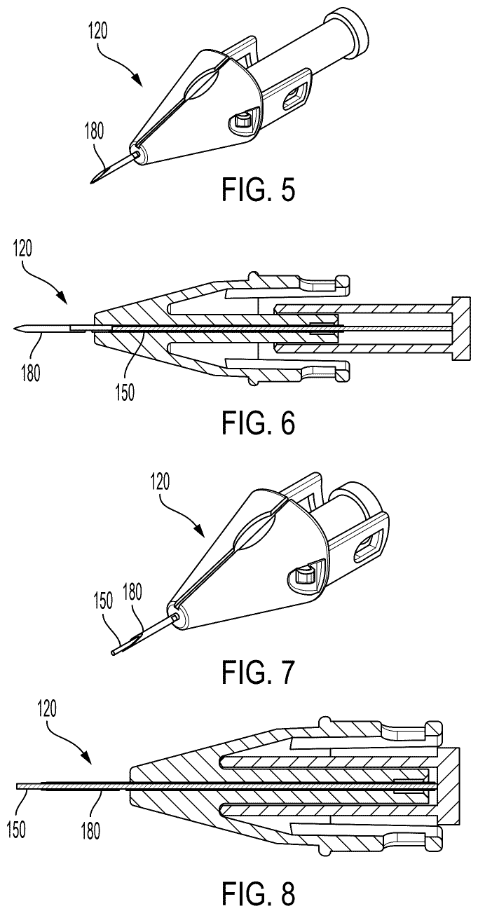

FIGS. 5 through 8 show the insertion tip 120 before and after depression of the push button 140. FIGS. 5 and 6 show the inserter tip 120 before activation of the insertion device 100 and before insertion of the implant into the eye. The pusher wire 150 is still fully disposed in the needle 180 of the insertion device 100. FIGS. 7 and 8 show the inserter tip 120 after activation of the insertion device 100 and insertion of the implant into the eye. The pusher wire 150 extends beyond the tip of the needle 180 at a predefined distance.

As shown in the FIGS. 5 through 8, the needle 180 may include a window 180a, such as an opening in the needle 180. A surgeon may use the window 180a of the needle 120 to visually confirm the presence of an implant in the insertion system 100. For example, as seen in FIGS. 5 and 6, the implant can be seen through the window before activation of the insertion system.

A surgeon may also use the window 180a to visually confirm deployment of the implant after depression of the push button. For example, as seen in FIGS. 7 and 8, if the surgeon sees the pusher wire 150 in the window 185, the surgeon knows the pusher wire propelled the implant out of the insertion device 100. In some embodiments, the pusher wire 150 is a bright color, to help the surgeon better differentiate the pusher wire 150 from the needle 180. In some embodiments, the insertion device provides the user another indication that the insertion device has been activated, such as tactile or auditory feedback.

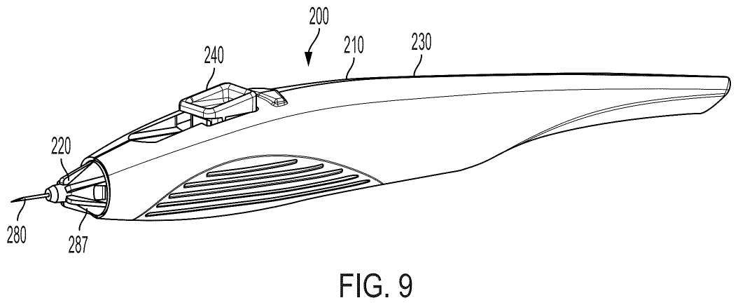

FIG. 9 shows an isometric view of an example insertion device 200, configured for delivery of an implant into the eye, according to another example of the present disclosure. At the outset, it should be noted that any of the features or aspects of insertion device 100 discussed above could, likewise, be implemented with insertion device 200; similarly, it should be noted that any of the features or aspects of insertion device 200 discussed below could, likewise, be implemented with insertion device 100. As shown in FIG. 9, the insertion device 200 includes a body 210 and an inserter tip 220. In some examples, the body 210 and the inserter tip 220 of insertion device 200 may be combined with or may be the same as any of the embodiments of the body 110 and the inserter tip 120 of insertion device 100.

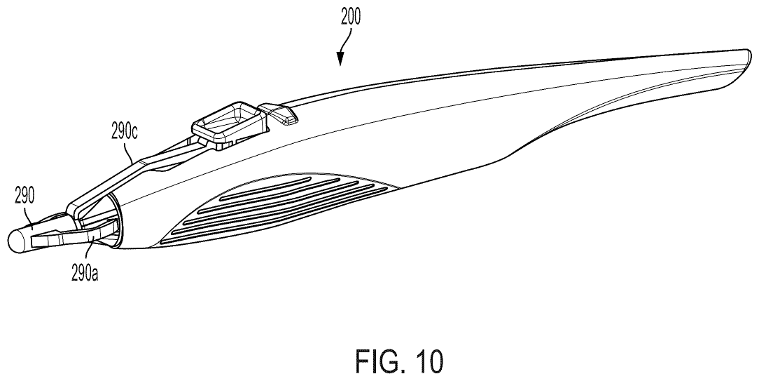

As shown in FIG. 10, in some examples, the insertion device 200 includes a protective cap 290 configured to protect the needle 280 and the inserter tip 220 during transportation and handling. The user removes the protective cap 290 from insertion device 200, prior to use. The protective cap 290 can be affixed to the inserter tip 220 or the body 210 by any fastening means.

Similar to protective cap 190, the protective cap 290 of the inserter tip 220 can act as a locking mechanism to prevent the push button 240 from inadvertent depression during transportation and handling. For example, as shown in FIG. 10, the protective cap 290 may include an arm 290c that rests under the push button 240, preventing the push button 240 from being depressed. Arm 290c extends along a length of insertion device 200 from push button 240 towards inserter tip 220. The protective cap 290 can also include one or more tabs 290a that engage with grooves along housing 230 or inserter tip 220. The user may squeeze or separate the tabs 290a for easier removal of the protective cap 290 from the inserter tip 220. Additionally, the tabs 290a of the protective cap 290 can include grooves or protrusions for improving the grip of the user during removal of the cap 290.

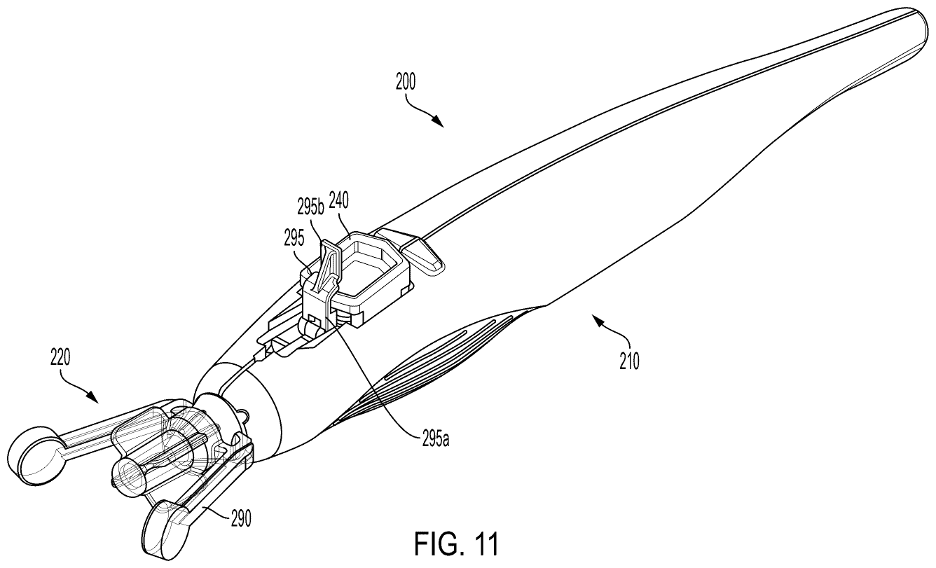

As shown in FIG. 11, in some examples, the insertion device 200 includes a protective cap 290 of the inserter tip 220 and a mechanism 295 for preventing inadvertent depression of the push button 240, separate from the protective cap 290. This can allow the inserter tip 220 to be protected during transportation and handling and prevention of depressing the push button 240 when the inserter tip 210 and the body 220 are separate. In some embodiments, the mechanism 295 for preventing push button 240 depression includes a notch 295a extending below the push button 240 and an arm (not shown) removably coupled to a portion of the body 210, such as the backbone. In some embodiments, the mechanism 295 includes a tab 295b for user interaction. The user may apply pressure to the tab 295a for removal of the mechanism 295 prior to the procedure. Namely, user pushes on tab 295a with a thumb, and pivots the entire mechanism 295 off of push button 240; the user can then freely depress bush button 240 (e.g., for activation of insertion device 200).

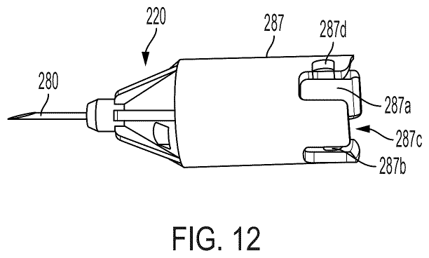

FIG. 12 shows an isometric view of the inserter tip 220 of the insertion device 200. Similar to inserter tip 120, the inserter tip 220 may include a needle 280 and a cover 287. The cover 287 of the inserter tip 220 may protect the components of the inserter tip 220. Additionally, as depicted, the cover 287 is configured to couple the inserter tip 220 to the body 210 of the insertion device 200. For example, the cover 287 of the inserter tip 220 may include one or more features configured to couple with one or more features of the body 210, such as the backbone of the body 210. In the illustrated embodiment, the cover 287 includes one or more slots 287a, 287b, 287c configured to couple with one or more protrusions on the body 210. Each slot (e.g., slot 287a) includes a notch (e.g., notch 287d) extending from a side of the slot 287a. In an example embodiment, the slots 287a-c are geometrically keyed, such that inserter tip 220 can only couple to body 210 in a particular orientation. It should be appreciated that the quantity of slots 287a- c may vary in alternative embodiments.

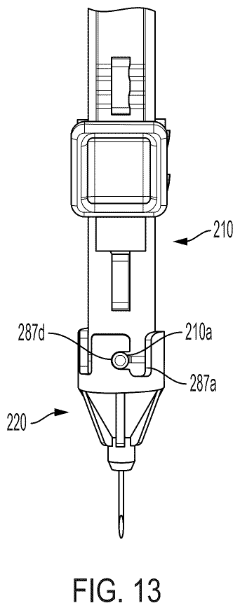

As depicted in FIG. 13, to secure the inserter tip 220 to the inserter body 210, the user aligns the protrusion 210a of the body 210 with the slot 287a and advances the inserter tip 220 so that protrusion 210a moves in an axial direction into the slot 287a. Once the protrusion 210a is disposed within the slot 287a, the user twists the inserter tip 220 relative to the body 210 (or vice versa) to secure the protrusion 210a in the notch 287d of the slot 287a. In an embodiment, notch 287d is configured to press fit with protrusion 210 upon twisting. If the user wishes to remove the inserter tip 220 from the body 210, the user can easily twist the inserter tip 220 to remove the protrusion 210a from the notch 287d and then move the inserter tip 220 axially away from the body 210. This configuration allows a user to easily secure and remove the inserter tip 220 from the body 210. However, it can be appreciated that other securing mechanisms can be used to attach the inserter tip 220 to the body 210.

In some examples, the axial movement of the inserter tip 220 with respect to the body 210 engages the internal components of the body 210 to prepare or charge the insertion device 200 for use. For example, as will be discussed in more detail herein, when the inserter tip 220 pushes against the body 210, the inserter tip 220 compresses a spring within the body 210. The subsequent twisting of the inserter tip 220 locks the spring in a compressed state, causing the spring to store potential energy. When delivering the implant from insertion device 200, the internal mechanisms of the insertion device 200 cause this potential energy to transfer to kinetic energy, which ultimately propels the implant into the eye. This configuration allows a user to reuse the body 210 with multiple inserter tips 220 depending on the type or dose of pharmaceutical.

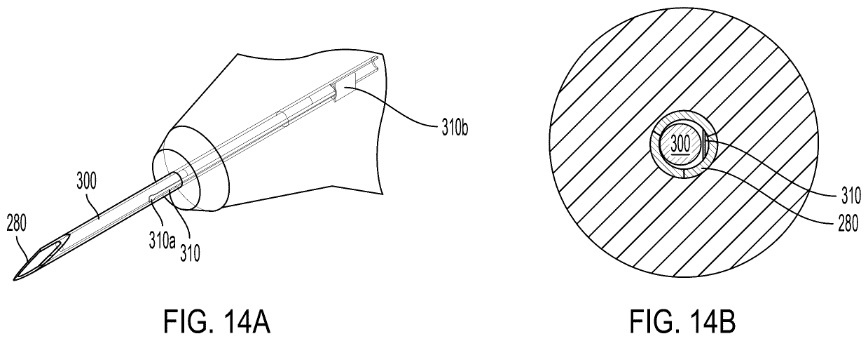

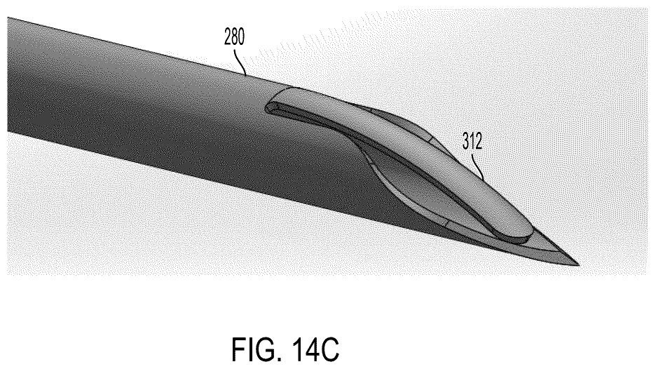

As depicted in FIG. 14A, prior to use of the insertion device 200, one or more implants 300 are loaded in the needle 280 of the inserter tip 220. To prevent movement of the implant 300 after it is positioned within the needle 280, a retaining member 310 is disposed within needle 280 to hold the implant 300 into place within the needle 280. For example, the implant 300 may generally have a diameter smaller than the internal diameter of the needle 280. This allows a user or manufacturer to easily load the implant 300 into the needle 280; but, this configuration provides clearance between the implant 300 and an internal surface of the needle 280. The retaining member 310 minimizes the clearance between the implant 300 and the internal surface of the needle 280. The retaining member 310 may be composed of thin layer polyimide, stainless steel, or other suitable material. In some examples, the retaining member is flexible.

In some examples, the retaining member 310 includes a thin, elongate body with at least one tab 310a at the distal end of the retaining member 310. As illustrated in FIG. 14B, the tab 310a contacts the side surface of implant 300, pressing the implant 300 into an internal wall of the needle 280, and thus creating an interference or friction fit between the needle 280 and the implant 300. In some examples, the tab 310a has a generally triangular shape, a hook-like shape, or any suitable shape.

In an example embodiment, needle 280 includes additional or alternative features for retention of implant 300. For example, as shown in FIG. 14C, needle 280 may include a pivotable flap 312, affixed to a distal end of needle 280. In an embodiment, pivotable flap 312 is formed integrally with needle 280 (e.g., continuous material); in a different embodiment, pivotable flap 312 is coupled to needle 280 (e.g., as a separate component). It should be appreciated that pivotable flap 312 could be the same, or different, material as that of needle 280. For example, pivotable flap 312 could be constructed of a stainless steel, another metal such as copper, a polymer, a rubber, or other similar materials. Materials selection can dictate the overall flexibility of flap 312. The pivotable flap 312 may extend from a top surface of the needle 280 to an inner surface of the needle 280. In some embodiments, the pivotable flap 312 contacts an inner surface of the needle 280 about 180 degrees from the top surface from which the pivotable flap 312 extends.

Responsive to a threshold force, such as the delivery force of implant 300, flap 312 is configured to pivot outward (i.e. away from the inner surface), thus permitting implant 300 to be ejected from the distal end of needle 280. Beneficially, flap 312 is configured such that any force below the threshold force is insufficient to pivot the flap 312 outward. Namely, flap 312 helps to physically retain implant 300 from unintentionally falling out of needle 280 (i.e., during shipment or handling). Flap 312 may be configured to pivot at a given force based off material selection and/or overall geometry. For example, a smaller cross-section of flap 312 will result in a lower threshold force to pivot; a larger cross-section of flap 312 will result in a higher threshold force to pivot.

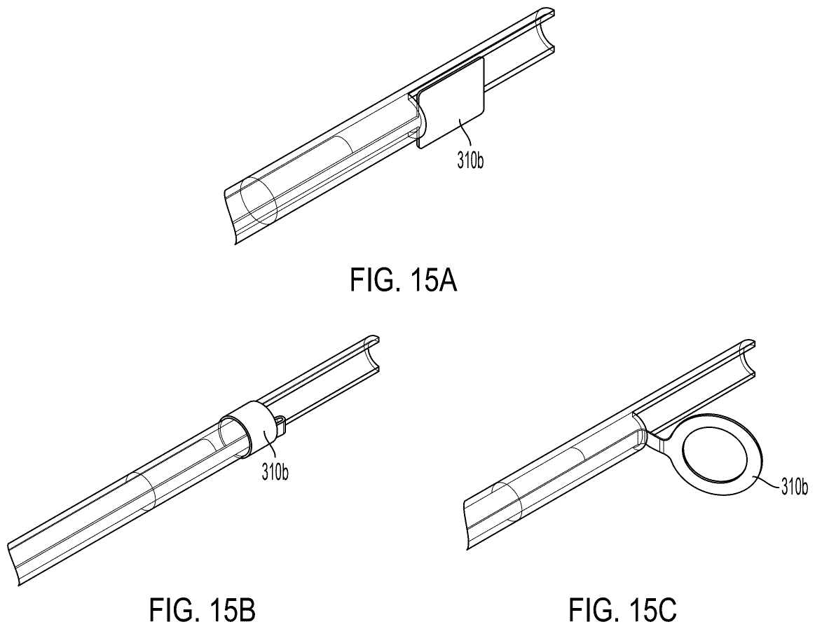

In certain embodiments, a user inserts the retaining member 310 into the needle 280 from the distal end of the needle 280 and advances the retaining member 310 along the length of the needle 280; in other embodiments, the retaining member 310 is inserted into needle 280 during manufacturing. The distal end of the retaining member 310 may include a feature used to anchor the retaining member 310 to the needle 280 to ensure proper securement of the implant 300 in the needle 280. FIGS. 15A through 15C show different configurations of features at the distal end of the retaining member 310 according to examples of the present disclosure. As depicted in FIG. 15B, in some examples, the feature 310b at the distal end of the retaining member 310 includes a collar configured to surround the outer diameter of the needle 280. As depicted in FIGS. 15A and 15C, in other examples, the feature 310b at the distal end of a retaining member 310 includes a circular or a rectangular tab with a width greater than an inner diameter of the needle 280. This prevents further axial movement of the retaining member 310 once the distal end of the retaining member 310 is disposed adjacent to the proximal end of the needle 280.

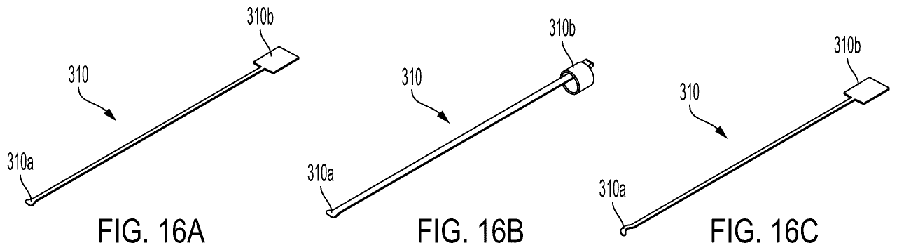

FIGS. 16A through 16C illustrate different embodiments of the retaining member 310 according to examples of the present disclosure, similar to those in FIGS. 15A through 15C. For example, the proximal end 310a of the retaining member 310 may be flat with a width greater than the shaft of the retaining member 310 as shown in FIGS. 16A and 16B or may curved as shown in 16C. In some embodiments, the distal end 310b of the retaining member 310 may be flat, such as a flat geometrical shape shown in FIGS. 16A and 16C, or may be a collar as shown in FIG. 16C.

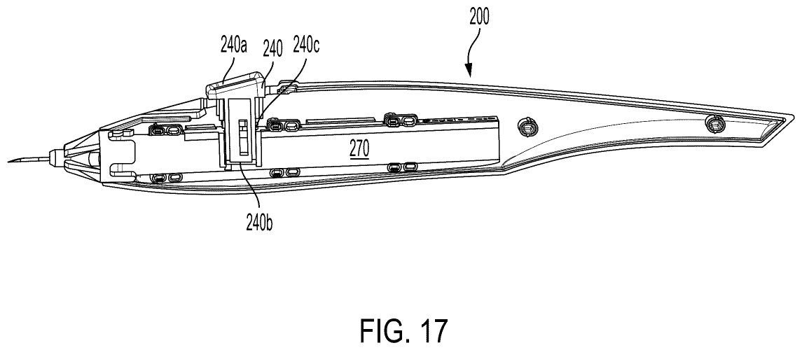

FIG. 17 shows a side view of the internal components of the insertion device 200 with the insertion tip 220 secured to the body 210. Similar to insertion device 100, the body 210 of insertion device 200 includes an external housing 230 configured to protect the components contained therein, a backbone 270, a pushing mechanism 260, a push button 240, and a pusher wire 250. The push button 240 and the pushing mechanism 260 may mount to the backbone 270 of the body 210. For example, the backbone 270 may include one or more features configured to couple with the push button 240 and/or the pushing mechanism 260. In the depicted embodiment, the backbone 270 includes a generally cylindrical member surrounding the pushing mechanism 260 and the pusher wire 250. The backbone 270 may include one or more protrusions configured to mate the inserter tip 220 to the body 210 of the insertion system 200. The use of backbone 270 may ensure that most of the precision engineering is isolated to one critical component of the insertion device 200.

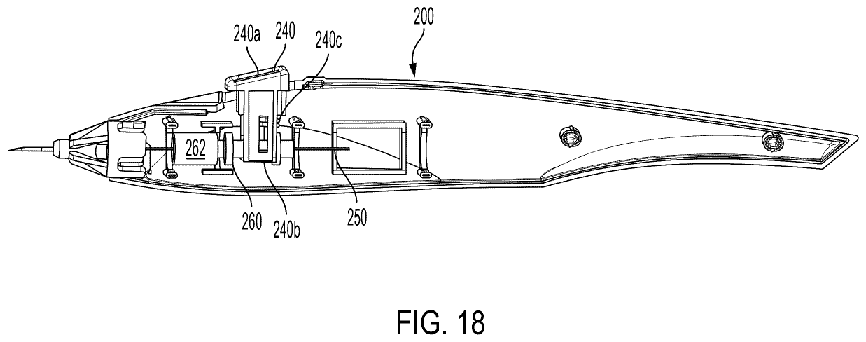

FIG. 18 is the insertion device 200 of FIG. 17 with the backbone 270 removed for visibility of the internal components. In some embodiments, the push button 240 may include a button 240a, a first arm 240b extending from the button 240a towards the backbone 270, and a spring 240c disposed between the button 240a and a top surface of the backbone 270. The spring 240c biases the push button 240 in an upward position. For example, spring 240c is disposed in the same general direction that the button 240a is permitted to translate.

Generally, the button 240a is configured to be depressed by a user, such as via a user's thumb or finger. Before depression of the button 240a, the insertion device 200 is generally disposed in an inactivated state as shown in FIGS. 17 and 18. When the user depresses the button 240a and compresses the spring 240c, the insertion device 200 activates, propelling the pushing mechanism 260 and pusher wire 250 forward, as will be discussed in more detail herein. Once the user removes the force exerted on the button 240a, the spring 240c biases the push button 240a upward, returning the push button 240 back to the upward biased position.

In the illustrated embodiment, the pushing mechanism 260 includes a shuttle 262 and a spring (not shown), with the spring coupled to one end of the shuttle 262. In the inactivated state, the spring is compressed, thus storing potential energy. For example, the spring may be compressed against a surface of the backbone 270 and an end of the shuttle 262.

As illustrated in FIGS. 17 and 18, the first arm 240b may extend from the button 140a and may engage with the pushing mechanism 260 through a slot in the backbone 270. A portion of the shuttle 262 may include one or more features configured to engage with a feature on the first arm 240b of the push button 240, preventing axial movement of the shuttle 262 and holding the spring in a compressed state. For example, the shuttle 262 includes one or more grooves, such as around the circumference of the shuttle. The first arm 240b includes a protrusion that fits into the one or more grooves of the shuttle 262.

When a user depresses the push button 240, the first arm 240b moves with respect to the shuttle 262, disengaging the protrusion from the groove in the shuttle 262, allowing the shuttle 262 to move in an axial direction, thus activating the pushing mechanism 260. Activation of the pushing mechanism 260 results in a (partial or full) release of stored potential energy via the compressed spring. It can be appreciated that other mechanisms for storing potential energy can, alternatively, be used in place of the spring. For example, the pushing mechanism 260 may include one or more springs, flexing components, and/or gears to store potential energy and, when activated, release (at least a portion of) the potential energy as kinetic energy.

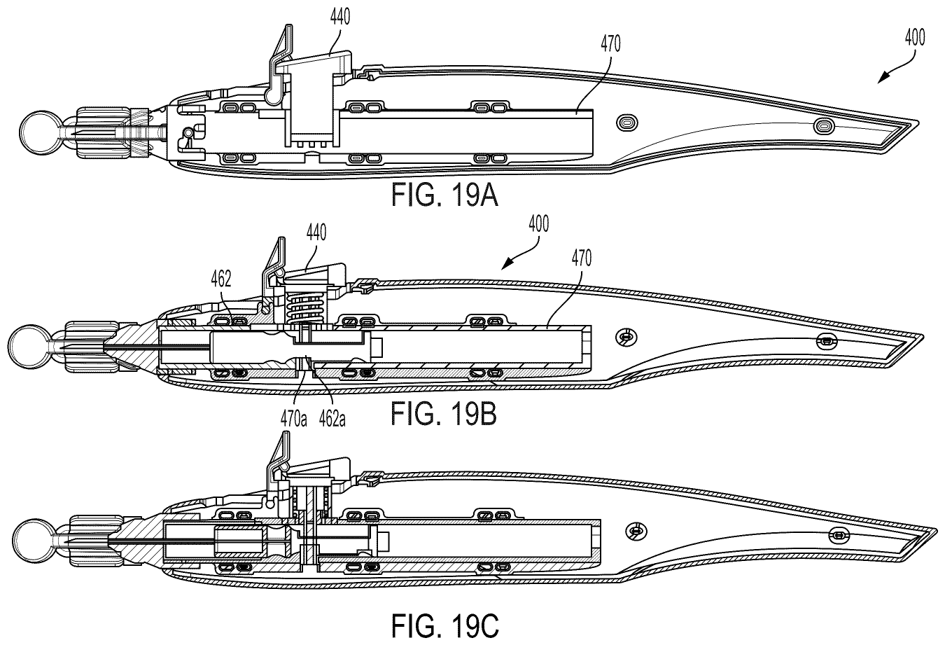

FIGS. 19A through 19C illustrate an internal view of an example insertion device 400, similar to insertion device 200, with modified internal components. Insertion device 400 may insert a single implant 300 into the eye of a patient. As shown in FIGS. 19B and 19C, a portion of the push button 440 may extend through a channel in the shuttle 462. The shuttle may also include an additional channel extending from the first channel that allows the shuttle 462 to move with respect to the push button 440. In some embodiments, the first channel is perpendicular to the additional channel. The backbone 470 and/or the shuttle 462 may include a recess configured to receive the portion of the push button 440. Upon depression of the push button 440, the push button 440 may disengage from the recess 462a in the shuttle 462 allowing the shuttle 462 (and the pusher wire) to traverse forward.

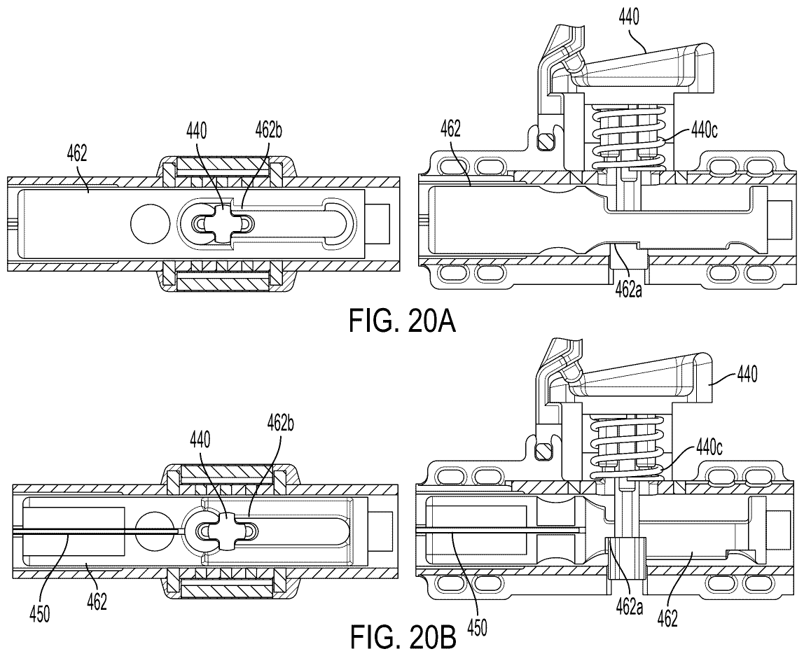

FIGS. 20A and 20B illustrate a bottom view and a close-up view of the shuttle 462 and push button 440 of insertion device 400. As illustrated in the figures, in an inactivated state, an end of the push button 440 may be positioned within a recess 462a of the shuttle 462, allowing the spring 440c to bias the push button 440 in an upward position. Upon depression of the push button 440, the push button 440 may disengage from the recess 462a, allowing the shuttle 462 to propel forward so that the bottom portion of the push button 440 travels through channel 462b of the shuttle 462. As shown in the depicted embodiment, the shuttle 462 may include a channel for receiving the pusher wire 450 so that movement of the shuttle 462 results in movement of the pusher wire 450.

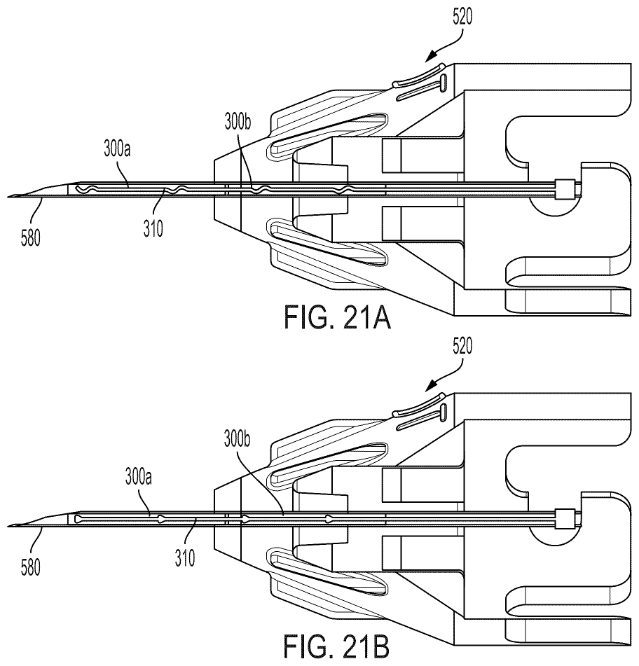

As described previously, the insertion system (100, 200, 400, etc.) described herein may be used to insert one or more implants 300 into the eye. FIGS. 21A and 21B illustrate a cross-sectional view of an inserter tip 520 with two implants 300a, 300b loaded into the needle 580. The implants 300a, 300b may be held in place by a retaining member 310 as described previously. In the depicted embodiments, the retaining member 310 may include one or more grooves (FIG. 21A) or one or more tabs (FIG. 21B) along the length for securing the implants 300a, 300b within the needle 580. The inserter tip 520 with multiple implants 300a, 300b may be used for inserting multiple implants into a patient's eyes.

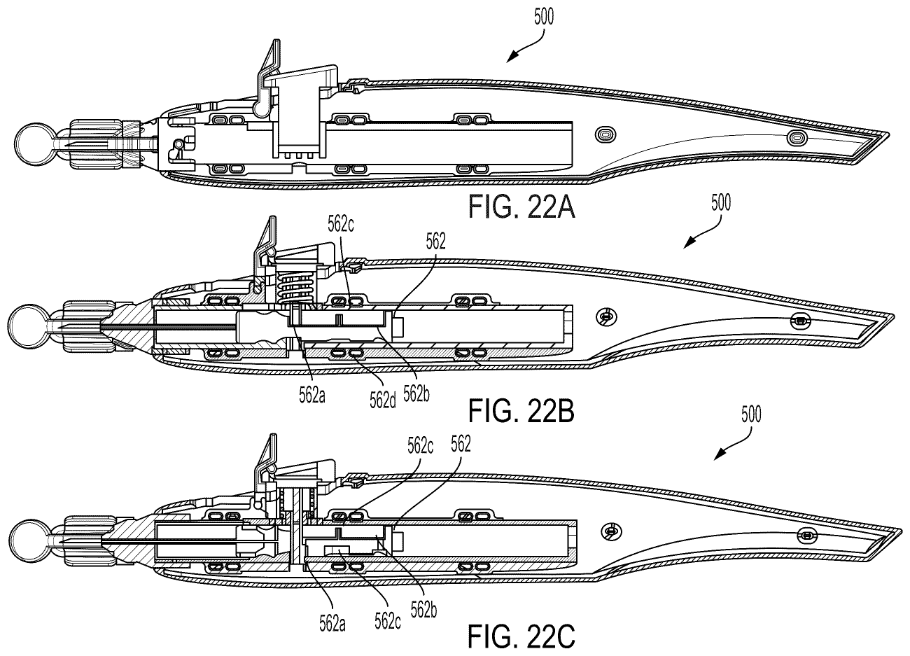

FIGS. 22A through 22C illustrate the internal components of insertion device 500 configured to deliver multiple implants into the eye of the patient. Insertion device 500 is similar to insertion devices 100, 200, 400, except the shuttle is modified to allow for multiple releases. For example, as shown in FIGS. 22B and 22C, shuttle 562 may include two portions: a first portion 562a for releasing the first implant and a second portion 562b for releasing the second implant. The shuttle 562 may include a post 562c between the first portion 562a and the second portion 562b to prevent axial movement of the shuttle 562 after deployment of the first implant. In some embodiments, the shuttle 562 includes a recess 562c between the first portion 562a and the second portion 562b. The push button 540 is configured to engage the recess 562c after the device releases the first implant and before the device releases the second implant.

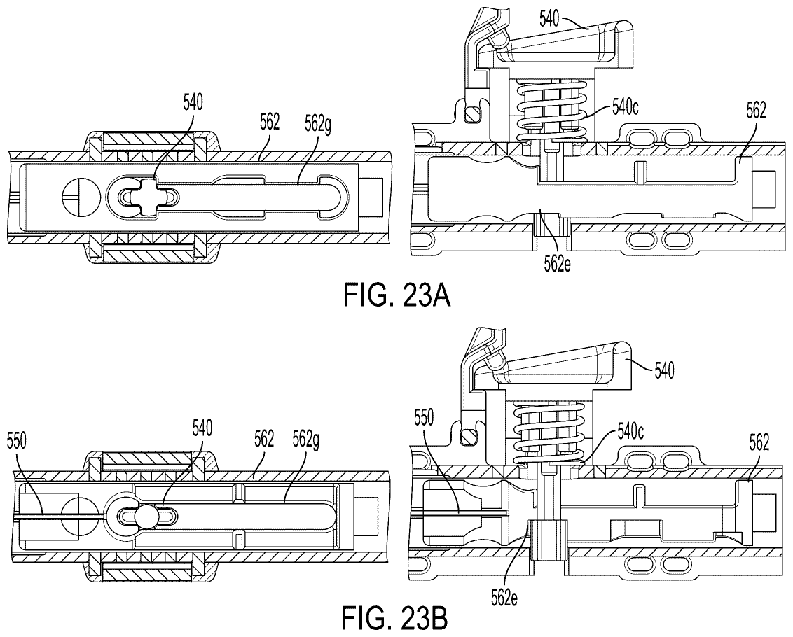

FIGS. 23A and 23B illustrate a bottom view and a zoomed in view of the insertion device 500 for deployment of multiple implants. As illustrated in the figures, in an inactivated state, an end of the push button 540 may be positioned within a first recess 562e of the shuttle 462, allowing the spring 540c to bias the push button 540 in an upward position. Upon depression of the push button 540, the push button 540 may disengage from the recess 562e, allowing the shuttle 562 to propel forward so that the bottom portion of the push button 540 travels through channel 562g of the shuttle 562. As shown in the depicted embodiment, the shuttle 462 may include a channel for receiving the pusher wire 550 so that movement of the shuttle 562 results in movement of the pusher wire 550.

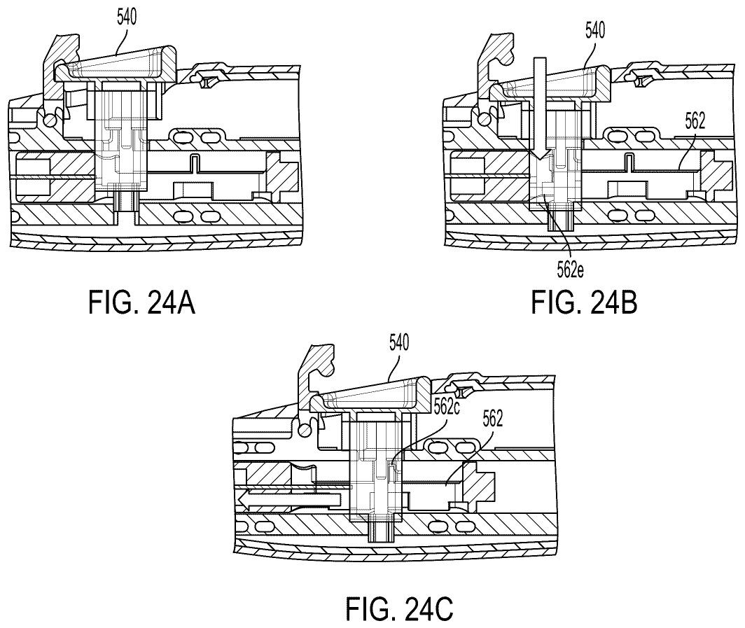

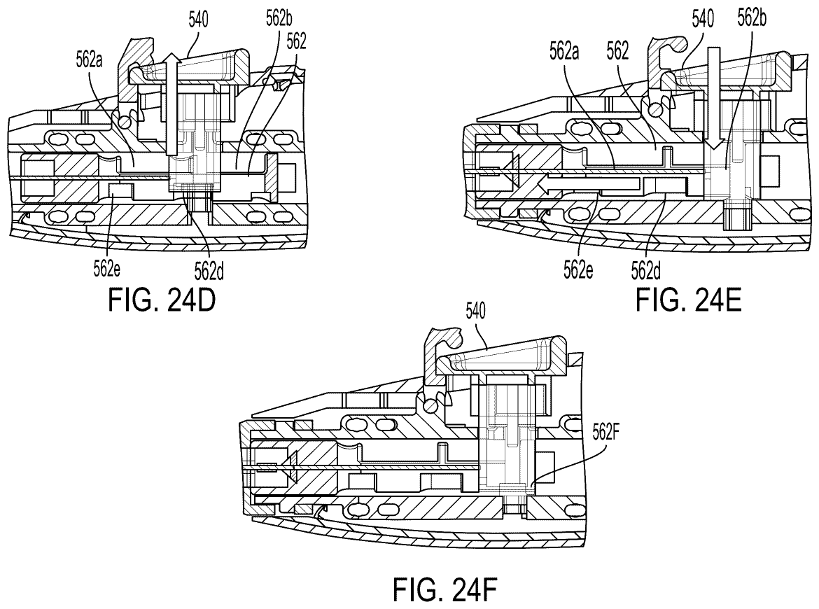

FIGS. 24A through 24F illustrate the process undergone by the insertion device 500 for deploying multiple implants. FIG. 24A illustrates the insertion device in the inactivated state with the push button 540 biased upward. FIG. 24B illustrates depression of the push button 540 in the direction of arrow. The force from the user depressing the push button 540 the first time disengages the push button 540 from the recess 562e in the shuttle 562 causing a portion of the potential energy stored in the spring to translate into kinetic energy. The release of energy traverses the shuttle 562 forward in the direction of the arrow deploying the first implant as illustrated in FIG. 24C. The shuttle 562 traverses forward until the push button 540 contacts the post 562c between the first portion 562a of the shuttle and the second portion 562b of the shuttle 562. The push button 540 then engages with the second recess 562d between the first portion 562a and the second portion 562b of the shuttle 562, which biases the push button 540 back up and into an inactivated state ready for a second depression as shown in FIG. 24E.

Depressing the push button 240 the second time disengages the push button 540 from the second recess 562d in the shuttle 562 causing another portion of the potential energy stored in the spring to translate into kinetic energy. The additional release of energy traverses the shuttle 562 forward deploying the second implant as illustrated in FIG. 24E. After deployment of the second implant, the push button 540 engages with a third recess 562f at an end portion of the shuttle 562, which again biases the push button 540 back up and into an inactivated state as shown in FIG. 24F. While the shuttle 562 includes two portions 562a, 562b for delivering two implants, it can be appreciated that the insertion device may be modified to include three, four, or more portions to deliver three, four, or more implants.

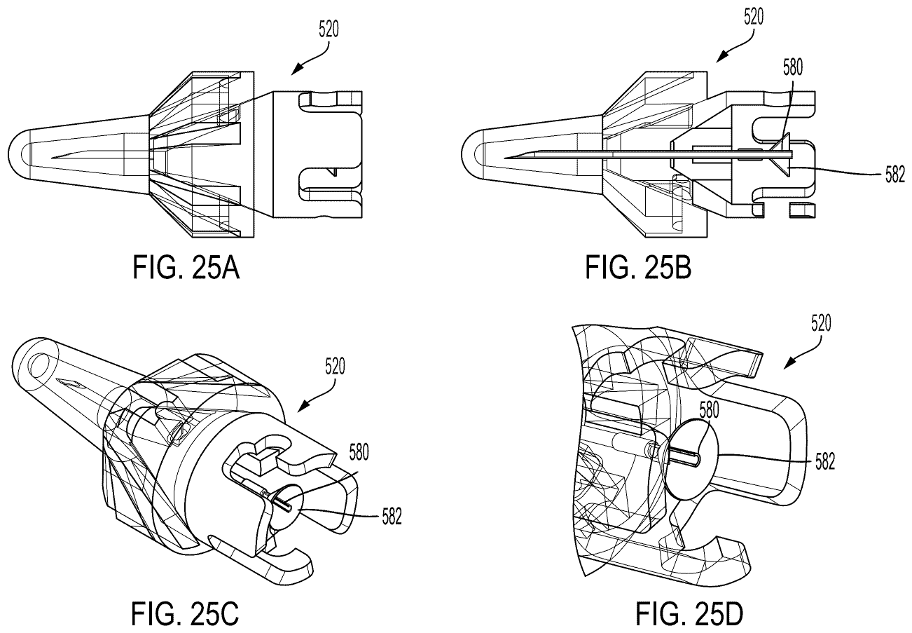

FIGS. 25A through 25D illustrate an inserter tip 520 according to another example of the present disclosure. Inserter tip 520 may be used with an inserter device according to an example of the present disclosure (100, 200, 400, etc.). In an embodiment, a proximal end of the needle 580 may include a funnel 582 or other alignment mechanism. When affixing the inserter tip 520 to the body, the funnel 582 may assist with pusher wire 550 alignment with respect to the needle 520. For example, when the inserter tip 520 is affixed to the body, the pusher wire 550 may contact an inside surface of the funnel 582, guiding the pusher wire into a channel in the needle. In some embodiments, a portion of the proximal end of the needle 580 is removed, creating a slot through which the pusher wire 550 may be side loaded into the channel in the needle 580.

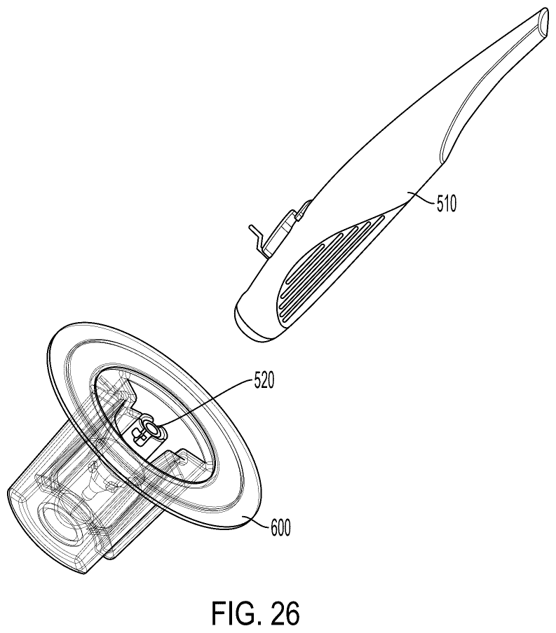

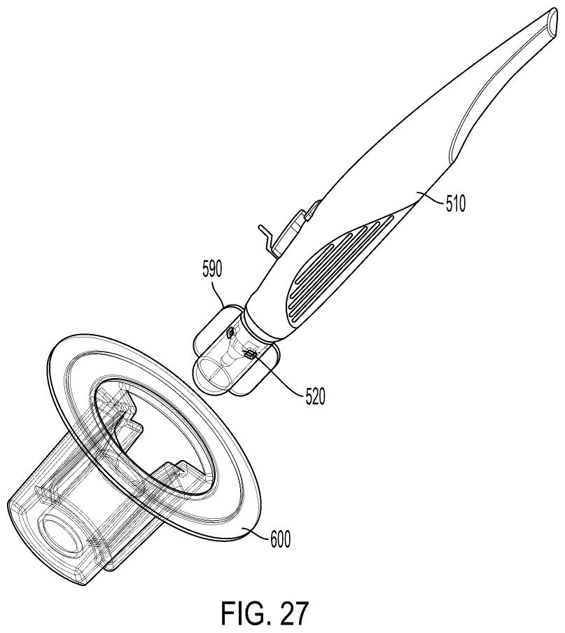

As discussed previously, in some embodiments, the inserter tip 520 is packaged separately from the body 510 during the manufacturing process. FIG. 26 illustrates the inserter tip 520 packaged in a different packaging than the body 510. In some embodiments, the inserter tip 520 is packaged in a sterile tray 600. The body 510 may attach to the inserter tip 520 while the inserter tip 520 is still in the packaging. This may keep the needle sterile prior to use and prevent accidental needle stick before the procedure. To connect the body 510 and the inserter tip 520, a user may move the body 510 into the end of the inserter tip 520 exposed from the packaging. Then, the user may rotate the body 510, such as a quarter turn or half turn, to lock the inserter tip 520 onto the body 510. As illustrated in FIG. 27, after the inserter tip 520 and body 510 are locked together, the user may remove the inserter tip 520 with the body 510 from the packaging. As discussed previously, the inserter tip 520 may include a protective cap 590. Prior to the start of the procedure, the user removes the protective cap 590 to expose the needle of the insertion device. Protective cap 590 and/or sterile tray 600 may be colored, depending on the type or dose of implant(s) or pharmaceutical(s) loaded into to the inserter tip 520. For example, one type of implant may have a blue cover indicating a first drug or dosage of drug, while another type of implant may have a red cover indicating a second different drug or dosage of drug. This helps prevent the surgeon from accidentally implanting the wrong pharmaceutical into the eye.

Packaging the inserter tip separate from the body may be beneficial for multiple reasons. First, the inserter tip may be preloaded with an implant, which may require refrigerated storage. Keeping the implant separate from the body may save on storage space since only the inserter tip containing the implant will need to be stored in the refrigerator. Additionally, multiple inserter tips may be used with the same body. The inserter tip or the packaging of the inserter tip may be colored depending on the type of pharmaceutical of the implant. It may be easier for a user to determine right before the procedure what type of pharmaceutical is needed and attach the inserter containing the specific pharmaceutical to the body.

In one example, the implant delivers active pharmaceutical agents to the eye. In some embodiments, pharmaceutical agents include multikinase inhibitors (MKIs) such as, and not limited to, axitinib (INLYTA®), dasatinib (SPRYCEL®), erlotinib (TARCEVA®), imatinib (GLIVEC®), nilotinib (TASIGNA®), pazopanib (VOTRIENT®), sunitinib (SUTENT®), cabozantinib, lenvatinib, regorafenib, sorafenib, and vandetinib. In some embodiments, the pharmaceutical agent may be one or more Transiently Acting Silencing RNAs (tasiRNAs). In some embodiments,

MKIs have been shown to inhibit vascular leakage clinically in the treatment of wet age-related macular degeneration (wAMD), retinal vein occlusion (RVO) and diabetic macular edema (DME). The approved anti-VEGFs are effective; however, their short duration a creates burden on the practice and patient. The inserts described herein provide a long-acting intravitreal release that would be effective in managing the disease for approximately 6 to 12 months.

The tolerability and efficacy of MKI intravitreal implant, to inhibit retinal vascular leakage was investigated in a DL-AAA Dutch-Belted rabbit model. DL-AAA rabbits were induced 8 weeks prior to study start. Seven DL-AAA female rabbits received 1 implant OD (left eye) and 1 placebo implant OS (right eye), and 3 received 2 implants OU (both eyes). Eyes were monitored using fluorescein angiography (FA), aide Angle (55°) IR imaging, and slit lamp ophthalmic exams including McDonald-Shadduck scoring at Days 1, 8 and monthly thereafter for 12 months. To assess tolerability, 3 naïve female Dutch Belted rabbits received 1 implant OD and sham injection OS, and 3 rabbits received 2 implants OD and 2 placebo implants OS. Plasma was collected to monitor systemic drug exposure.

Administration of 1 or 2 implants showed inhibition of retinal vascular leakage compared to placebo on FAs beginning at Day 8 and continuing for 12 months. Both 1 and 2 implants provided maximal inhibition of vascular leakage. Observations consistent with intravitreal injections were observed and fully resolved. Systemic exposure was minimal, with values near or below the lower limit of quantitation. Initial signs of implant biodegradation were observed beginning on Day 210. Accordingly, long lasting inserts can offer a long duration therapeutic alternative in the treatment of wAMD, RVO and/or DME.

In some embodiments, an implant described herein comprises an MKI and a polymer such as poly(D,L-lactic acid) (PLA), poly(D,L-lactic-co-glycolic acid) (PLGA), or a combination thereof.

The implant can remain in the eye for six months or longer. The insertion device 100 can accommodate implants of different sizes and shapes. In one example, the size of the implant is controlled relative to the pusher wire 150 as to prevent air bubbles in the delivery of the implant. In one example, the implant or the pusher wire 150 may include patterning or surface finishes configured to prevent adhesion of the pusher wire 150 to the implant during insertion of the implant. The patterning or surface finishes can be laser machined, traditionally machined, obtained by surface texturing method, EDM based, or any added by suitable method.

The implant can be any suitable shape, for example a cylinder. In some embodiments, the implant may comprise a rod, a tube, or a modified bar design with custom shaping to ensure proper drug interaction. The implant can be any suitable size. For example, the implant can have an outer diameter of about 0.35 mm and a length of about 6 mm.

One embodiment comprises a method of delivering an implant into the eye. The method includes inserting a needle 180 of the insertion device (i.e., 100, 200, 400, 500) of any of the examples described herein into the eye. In some embodiments, a surgeon inserts the needle 180 into the posterior chamber of the eye. The method may further include depressing the push button 140 to deliver the implant 200 to the eye. In some examples, the method includes repositioning the insertion device 100 after delivery of a first implant 300a into the eye. The method further includes depressing the push button 140 a second time to deliver a second implant 300b to the same or different eye. In some examples, the method includes removing the inserter tip 120 from the body 110 after use, resetting the internal components of the body 110, and securing a different inserter tip 120 to the body 110 for an additional use. In one example, the method further comprises affixing the inserter tip 120 to the body 110 prior to inserting the needle 180 into the eye. In one example, the method comprises loading the insertion device 100 with the implant. In one example, the method comprises a method of treating a disease of the eye.

The above description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. An insertion device for injecting an implant into an eye, the insertion device comprising:
a body comprising:
a push button comprising a button, a first spring, and an arm extending from the button, wherein the first spring biases the arm and the button in an upward position,
a pusher wire, and
a pushing mechanism comprising a shuttle and a second spring,
wherein the pusher wire comprises a first end and a second end and wherein the second end of the pusher wire is coupled to the shuttle; and
an inserter tip comprising a needle, wherein the inserter tip is removably coupled to the body,
wherein, upon depression of the push button, the pushing mechanism is configured to displace the pusher wire, such that a portion of the pusher wire translates through the needle.

2. The insertion device of claim 1, wherein the pusher wire translates through the needle at a consistent velocity, wherein the consistent velocity is independent of a force applied to the push button via depression of the push button.

3. The insertion device of claim 1, wherein the body further comprises a backbone, wherein the push button and the pushing mechanism are integrated with the backbone.

4. The insertion device of claim 1, wherein the shuttle comprises a first groove and a second groove, wherein the first groove and the second groove are configured to receive a portion of the arm of the push button.

5. The insertion device of claim 4, wherein the insertion device comprises a first inactivated state where the portion of the arm is in the first groove, a first active state where the portion of the arm disengages from the first groove, and a second inactivated state where the portion of the arm is in the second groove.

6. The insertion device of claim 1 further comprising:
a retaining member; and
an implant disposed within the needle,
wherein the retaining member exerts a force on the implant, holding the implant in place within the needle.

7. The insertion device of claim 6, wherein depression of the push button causes the portion of the pusher wire to translate through the needle and into the implant, wherein a force exerted on the implant from the pusher wire is greater than the force from the retaining member, causing the implant to eject from the needle.

8. The insertion system of claim 6, wherein the implant comprises a pharmaceutical for delivery to the eye.

9. An insertion tip configured to be removably coupled to an insertion device for injecting an implant into an eye, the insertion tip comprising:
a needle; and
a cover for receiving the needle,
wherein the cover comprises one or more slots,
wherein the one or more slots comprise a notch on a side surface of the slot, and
wherein the one or more slots are configured to secure the insertion tip to the insertion device.

10. The insertion tip of claim 9, wherein the needle comprises a double beveled edge.

11. The insertion tip of claim 9, wherein the inserter tip comprises the implant configured to be injected into the eye.

12. The insertion tip of claim 11, wherein the implant is disposed within the needle.

13. The insertion tip of claim 12 further comprising a retaining member configured to exert a force on the implant into an internal surface of the needle.

14. The insertion tip of claim 13, wherein the retaining member comprises a distal end portion, a proximal end portion, and an elongate member between the distal end portion and the proximal end portion, wherein the distal end portion is configured to exert the force on the implant into the internal surface of the needle.

15. The insertion tip of claim 14, wherein the proximal end portion of the retaining member is configured to anchor the retaining member to the needle, and wherein the elongate member is disposed within the needle.

16. The insertion tip of claim 11, wherein the implant is a first implant, and wherein the insertion tip further comprises a second implant.

17. The insertion device of claim 16, wherein the first implant and the second implant are disposed in series within the needle.

18. A method of inserting an implant into an eye, the method comprising:
inserting a needle of an insertion device into the eye, wherein the insertion device comprises a body removably coupled to an insertion tip, wherein the body comprises a push button, a pusher wire, and a pushing mechanism and wherein the insertion tip comprises a needle; and
depressing the push button of the insertion device, such that the implant is delivered through the needle of the insertion tip and into the eye at a consistent velocity.

19. The method of claim 18, further comprises loading the implant into a distal end of the insertion device prior to inserting the needle into the eye.

20. The method of claim 18, further comprising affixing the insertion tip to the body prior to inserting the needle of the insertion device.
